(19) United States
(12) Patent Application Publication
(10) Pub. No.: US 2002/0081579 A1
(43) Pub. Date: Jun. 27, 2002

(54) METHOD FOR THE ISOLATION OF NOVEL ANTIGENS

(75) Inventors: YASIR A.W. SKEIKY, SEATTLE, WA (US); DAVIN C. DILLON, REDMOND, WA (US); MARK R. ALDERSON, BAINBRIDGE ISLAND, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Jane E. R. Potter, Seattle, WA

(21) Appl. No.: 09/023,588

(22) Filed: Feb. 13, 1998

(51) Int. Cl. C12Q 1/68

(52) U.S. Cl. 435/6

(57) ABSTRACT

Methods for isolating novel antigens that stimulate CD4-positive T cells are disclosed. The methods provided comprise the steps of: transforming a host cell with a plasmid suspected of containing such a DNA sequence encoding such an antigen and inducing antigen expression; incubating the transformed host cell with at least one dendritic cell for a period of time sufficient to form a peptide/MHC Class II complex on the dendritic cell, the peptide being derived from the expressed antigen; incubating the dendritic cell with CD4+ T cells; determining the level of CD4+ T cell stimulation, thereby identifying a transformed host cell that expresses at least one CD4+ T cell-stimulating antigen; and isolating DNA from the transformed host cell. DNA sequences isolated using such methods, together with antigens encoded by such DNA sequences, are also provided.

### TECHNICAL FIELD

The present invention relates generally to methods for the isolation of novel antigens. The invention is more particularly related to methods for isolating antigens that stimulate T cells.

### BACKGROUND OF THE INVENTION

Both CD4+ and CD8+ T cells play a key role in the body's ability to mount an effective immune response to many disorders. For example, CD4+ T cells have been shown to be important in immunity to bacterial pathogens and also in mediating autoimmunity. While both CD4+ and CD8+ T cells recognize peptide/major histocompatability (MHC) complexes expressed on antigen-presenting cells wherein the peptide is derived from an antigen associated with a particular disorder, CD4+ T cells recognize peptide/MHC Class II complexes and CD8+ cells recognize peptide/MHC Class I complexes. The identification of expressed peptide/MHC Class I complexes has led to the isolation of several novel antigens that stimulate CD8+ T cells. However, the isolation of CD4+ T cell-stimulating antigens has been more problematic due, in part, to the large amounts of purified protein with which antigen-presenting cells must be incubated in order to obtain detectable CD4+ T cell responses, the small amounts of peptide/MHC II complex expressed by the antigen-presenting cells and the heterogeneous size of such complexes.

Sanderson et al. (J. Exp. Med. 182:1751-1757, 1995) describe a method for the isolation of antigens that stimulate CD4+ T cells wherein they first constructed a T cell hybrid containing a reporter gene by immunizing mice with Listeria monocytogenes (LM) to provide LM-specific CD4+ T cells which were subsequently used to form lacZ-inducible CD4+ T cell hybrids. An LM genomic DNA expression library was transformed into E. coli and, following antigen induction, the transformed E. coli incubated with syngeneic peritoneal macrophages to generate peptide/MHC class II complexes. The presence of peptide/MHC II complexes was detected by probing the macrophages with the lacZ-inducible LM-specific CD4+ T cell hybrids and staining with X-Gal substrate to visualize activated lacZ-positive T cells. The method of Sanderson et al. is technically complex and involves many steps, thereby being time-consuming and unsuitable for high-throughput use.

Accordingly, there is a need in the art for improved methods for isolating novel antigens that stimulate CD4+ T cells. The present invention fulfills these needs and further provides other related advantages.

### SUMMARY OF THE INVENTION

Briefly stated, this invention provides methods for identifying and isolating novel DNA sequences that encode CD4+ T cell-stimulating antigens, such methods being less technically difficult and less time-consuming than prior art methods, and more suitable for high-throughput use. In one aspect, such methods comprise the steps of: transforming a host cell with a plasmid suspected of containing such a DNA sequence and inducing antigen expression; incubating the transformed host cell with at least one dendritic cell for a period of time sufficient to form a peptide/MHC Class II complex on the dendritic cell, the peptide being derived from the expressed antigen; incubating the dendritic cell with CD4+ T cells and determining the level of CD4+ T cell stimulation, thereby identifying a transformed host cell that expresses at least one CD4+ T cell-stimulating antigen. In another aspect, the inventive methods further comprise isolating DNA from the transformed host cell.

The level of CD4+ T cell stimulation may be determined by measuring either cell proliferation or production of a cytokine, such as interferon-γ (IFN-γ). The inventive methods may be usefully employed to isolate novel antigens associated with, for example, infectious disease agents, tumor tissue and autoimmune disorders.

In one embodiment, the host cell employed in the inventive methods is selected from the group consisting of E. coli, yeast and mammalian cells. Preferably, a cDNA or genomic DNA library is first prepared from the agent or tissue of interest to provide the plasmids employed to transform the host cell.

In another aspect, methods for identifying novel DNA sequences that encode antigens comprising antibody epitopes are provided.

In a further aspect, the present invention provides DNA sequences isolated using the inventive methods, together with sequences that are complementary to such DNA sequences and sequences that hybridize thereto under conditions of moderate stringency. Expression vectors comprising such DNA sequences and host cells transformed therewith are also provided. In yet another aspect, the present invention provides polypeptides comprising amino acid sequences encoded by such DNA sequences. Such polypeptides may be usefully employed in the diagnosis and treatment of disorders such as infectious diseases, cancers and autoimmune disorders.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS

FIGS. 1A and 1B illustrate the presentation of E. coli expressed Tb38-1 by macrophages and dendritic cells, respectively, as measured by production of interferon-γ by an autologous Tb38-1-specific CD4+ T cell clone.

FIGS. 2A and 2B illustrate the presentation of E. coli expressed Tb38-1 by macrophages and dendritic cells, respectively, as measured by stimulation of T cell proliferation by an autologous Tb38-1-specific CD4+ T cell clone.

FIG. 3 illustrates stimulation of cell proliferation in three different CD4+ T cell lines by dendritic cells incubated with a single E coli colony expressing Tb38-1 mixed with either 0, 14 or 26 E. coli colonies expressing irrelevant genes.

FIGS. 4A and 4B illustrate the stimulation of proliferation and interferon-γ production, respectively, in T cells derived from a first PPD-positive donor (referred to as D7) by recombinant ORF-2 and synthetic peptides to ORF-2.

FIG. 5A and 5B illustrate the stimulation of proliferation and interferon-γ production, respectively, in T cells derived from a second PPD-positive donor (referred to as D160) by recombinant ORF-2 and synthetic peptides to ORF-2.

### DETAILED DESCRIPTION OF THE INVENTION

As noted above, the present invention is generally directed to methods for isolating DNA sequences that encode CD4+ T cell-stimulating antigens. As used herein the term “CD4+ T cell-stimulating antigens” refers to antigens that are capable of stimulating proliferation and/or cytokine production (for example, IL-2, GM-CSF, or IFN-γ) in T cells known to be CD4-positive. The inventive methods may be employed to isolate antigens associated with any disorder in which the stimulation of CD4+ T cells is believed to play a role in the body's immune response. For example, the methods may be used to isolate CD4+ T cell-stimulating antigens associated with infectious disease agents (such as Mycobacterium tuberculosis and Leishmaniasis), tumor tissue and autoimmune disorders.

A genomic or cDNA expression library is first prepared from the agent of interest, such as cultured M. tuberculosis or tumor tissue, using methods well known in the art (see, for example, Sambrook et al., Molecular Cloning: A Laboratory Manual, Cold Spring Harbor Laboratories, Cold Spring Harbor, N.Y., 1989). The resulting plasmids are transformed into a host cell and protein expression is induced, for example, with IPTG. In one embodiment, the host cell is selected from the group consisting of E. coli, yeast and mammalian cells. The transformed host cells are then incubated with dendritic cells for a period of time sufficient to form peptide/MHC Class II complexes. In general, the cells may be incubated for about 60 to about 90 minutes at a temperature of about 37° C. The dendritic cells employed in the inventive methods are preferably immature, monocyte-derived dendritic cells, as described in detail in Example 1. These cells have been shown to phagocytose particles including bacteria, such as E. coli.

The presence of peptide/MHC Class II complex is determined by measuring the level of CD4+ T cell stimulation. Preferably, the antigen-presenting dendritic cells are incubated with CD4+ T cells specific for the antigen of interest for periods of about 3 days at about 37° C. Such CD4+ T cells are generated by stimulation of T cells with antigen (for example, in the form of lysate or secreted proteins from an infectious agent, whole pathogen or tumor tissue) in the presence of antigen-presenting cells. CD4+ T cell lines of the desired specificity may be cloned using limiting dilution techniques well known in the art.

The level of T cell stimulation is determined by measuring cell proliferation and/or cytokine (for example, IL-2, GM-CSF or IFN-γ) production and comparing the level of stimulation to that obtained with control dendritic cells (i.e. dendritic cells presenting irrelevant antigens). As described in detail below, cell proliferation and cytokine production may be evaluated by methods well known in the art. For example cell proliferation may be determined by exposing cells to a pulse of radiolabeled thymidine and measuring the incorporation of label into cellular DNA. Cytokine production may be evaluated in an enzyme-linked immunosorbent assay (ELISA), wherein wells are scored positive if the readout is greater than 3 standard deviations above the mean of the control dendritic cells.

A positive readout indicates that the transformed host cell is expressing a CD4+ T cell stimulating antigen. DNA encoding the antigen may then be isolated from the transformed host cell using techniques well known in the art, such as those taught by Sambrook et al., Ibid.

The inventive methods may additionally be employed to identify antigens comprising antibody epitopes. The antibody response to protein antigens requires help from CD4+ T cells. These cells respond to specific antigens and produce cytokines and co-stimulatory molecules that stimulate antibody production by B cells. Such antibody production cannot occur in the absence of a CD4+ T cell response. Therefore, screening of antigens for CD4+ T cell recognition will yield proteins capable of stimulating helper T cell responses resulting in antibody production. Such protein antigens will be the target of an antibody response and are therefore good candidate serodiagnostic antigens. As described in detail below, the inventive methods may be used to isolate the M. tuberculosis antigen Tb38-1 which has been previously shown to react with sera from tuberculosis-infected patients.

The present invention also provides isolated DNA sequences selected from the group consisting of: a) sequences obtained using the inventive methods; b) sequence complementary to such sequences; and c) sequences that hybridize to the sequences of a) or b) under conditions of moderate stringency. Suitable moderately stringent conditions include prewashing in a solution of 5×SSC, 0.5% SDS, 1.0 mM EDTA (pH 8.0); hybridizing at 50° C.-65° C., 5×SSC, overnight or, in the case of cross-species homology at 45° C., 0.5×SSC; followed by washing twice at 65° C. for 20 minutes with each of 2×, 0.5× and 0.2×SSC containing 0.1% SDS.

Expression vectors comprising such sequences and host cells transformed with such vectors are also provided. Suitable host cells include prokaryotes, yeast and higher eukaryotic cells. Preferably, the host cells employed are E. coli, yeast or a mammalian cell line such as COS or CHO. The DNA sequences expressed in this manner may encode naturally occurring antigens, portions of naturally occurring antigens, or other variants thereof.

The present invention further provides polypeptides comprising an immunogenic portion of an antigen, or a variant of such an antigen that differs only in conservative substitutions and/or modifications, wherein the antigen comprises an amino acid sequence encoded by one of the above DNA sequences. The antigens (and immunogenic portions thereof) isolated using the methods described herein have the ability to induce an immunogenic response. More specifically, the antigens have the ability to induce proliferation and/or cytokine production (for example, IFN-γ and/or IL-2 production) in CD4+ T cells.

As used herein, the term “polypeptide” encompasses amino acid chains of any length, including full length proteins (i.e., antigens), wherein the amino acid residues are linked by covalent peptide bonds. Thus, a polypeptide comprising an immunogenic portion of one of the above antigens may consist entirely of the immunogenic portion, or may contain additional sequences. The additional sequences may be derived from the native antigen or may be heterologous, and such sequences may (but need not) be immunogenic.

“Immunogenic,” as used herein, refers to the ability to elicit an immune response (e.g., cellular) in a patient, such as a human, and/or in a biological sample. In particular, antigens that are immunogenic (and immunogenic portions or other variants of such antigens) are capable of stimulating cell proliferation and/or cytokine production comprising at least an immunogenic portion of one or more antigens may generally be used in the detection of or induction of protective immunity against the disorder with which the antigen is associated.

A “variant,” as used herein, is a polypeptide that differs from the native antigen only in conservative substitutions and/or modifications, such that the ability of the polypeptide to induce an immune response is retained. Such variants may generally be identified by modifying one of the above polypeptide sequences, and evaluating the immunogenic properties of the modified polypeptide using, for example, the representative procedures described herein.

A “conservative substitution” is one in which an amino acid is substituted for another amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his.

Variants may also (or alternatively) be modified by, for example, the deletion or addition of amino acids that have minimal influence on the immunogenic properties, secondary structure and hydropathic nature of the polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc domain.

Immunogenic portions of the antigens described herein may be prepared and identified using well known techniques, such as those summarized in Paul, Fundamental Immunology, 3d ed., Raven Press, 1993, pp. 243-247 and references cited therein. Such techniques include screening polypeptide portions of the native antigen for immunogenic properties. The representative proliferation and cytokine production assays described herein may generally be employed in these screens. An immunogenic portion of a polypeptide is a portion that, within such representative assays, generates an immune response (e.g., proliferation, interferon-γ production and/or interleukin-2 production) that is substantially similar to that generated by the full length antigen. In other words, an immunogenic portion of an antigen may generate at least about 20%, and preferably about 100%, of the cell proliferation induced by the full length antigen in the model proliferation assay described herein. An immunogenic portion may also, or alternatively, stimulate the production of at least about 20%, and preferably about 100%, of the interferon-γ and/or interleukin-2 induced by the full length antigen in the model assay described herein.

Portions and other variants of M. tuberculosis antigens may be generated by synthetic or recombinant means. Synthetic polypeptides having fewer than about 100 amino acids, and generally fewer than about 50 amino acids, may be generated using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin Elmer/Applied BioSystems Division, Foster City, Calif., and may be operated according to the manufacturer's instructions. Variants of a native antigen may generally be prepared using standard mutagenesis techniques, such as oligonucleotide-directed site-specific mutagenesis. Sections of the DNA sequence may also be removed using standard techniques to permit preparation of truncated polypeptides.

Recombinant polypeptides containing portions and/or variants of a native antigen may be readily prepared from DNA sequences isolated employing the inventive methods, using a variety of techniques well known to those of ordinary skill in the art. For example, supernatants from suitable host/vector systems which secrete recombinant protein into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify a recombinant protein.

In general, regardless of the method of preparation, the polypeptides disclosed herein are prepared in substantially pure form. Preferably, the polypeptides are at least about 80% pure, more preferably at least about 90% pure and most preferably at least about 99% pure. In certain preferred embodiments, described in detail below, the substantially pure polypeptides are incorporated into pharmaceutical compositions or vaccines for use in one or more of the methods disclosed herein.

The isolated DNA sequence obtained using the inventive methods (or a polypeptide comprising an amino acid sequence encoded by such a DNA sequence) may be employed in a patient to induce protective immunity against the disease agent from which the DNA sequence was isolated. As used herein, a “patient” refers to any warm-blooded animal, preferably a human. A patient may be afflicted with a disorder, or may be free of detectable disease and/or infection. In other words, protective immunity may be induced to prevent or treat a disorder.

In this aspect, the polypeptide is generally present within a pharmaceutical composition and/or a vaccine. Pharmaceutical compositions may comprise one or more polypeptides, and a physiologically acceptable carrier. Vaccines may comprise one or more of the above polypeptides and a non-specific immune response enhancer, such as an adjuvant or a liposome (into which the DNA molecule/polypeptide is incorporated). Such pharmaceutical compositions and vaccines may also contain other antigens, either incorporated into a combination polypeptide or present within a separate polypeptide.

Alternatively, a vaccine or pharmaceutical composition may contain DNA encoding one or more polypeptides as described above, such that the polypeptide is generated in situ. In such vaccines, the DNA may be present within any of a variety of delivery systems known to those of ordinary skill in the art, including nucleic acid expression systems, bacterial and viral expression systems. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface. In a preferred embodiment, the DNA may be introduced using a viral expression system (e.g., vaccinia or other pox virus, retrovirus, or adenovirus), which may involve the use of a non-pathogenic (defective), replication competent virus. Techniques for incorporating DNA into such expression systems are well known to those of ordinary skill in the art. The DNA may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

Routes and frequency of administration, as well as dosage, will vary from individual to individual. In general, the pharmaceutical compositions and vaccines may be administered by injection (e.g., intracutaneous, intramuscular, intravenous or subcutaneous), intranasally (e.g., by aspiration) or orally. Between 1 and 3 doses may be administered for a 1-36 week period. Preferably, 3 doses are administered, at intervals of 3-4 months, and booster vaccinations may be given periodically thereafter. Alternate protocols may be appropriate for individual patients. A suitable dose is an amount of polypeptide or DNA that, when administered as described above, is capable of raising an immune response in an immunized patient sufficient to protect the patient from the disorder for at least 1-2 years. In general, the amount of polypeptide present in a dose (or produced in situ by the DNA in a dose) ranges from about 1 pg to about 100 mg per kg of host, typically from about 10 pg to about 1 mg, and preferably from about 100 pg to about 1 μg. Suitable dose sizes will vary with the size of the patient, but will typically range from about 0.1 mL to about 5 mL.

While any suitable carrier known to those of ordinary skill in the art may be employed in such pharmaceutical compositions, the type of carrier will vary depending on the mode of administration. For parenteral administration, such as subcutaneous injection, the carrier preferably comprises water, saline, alcohol, lipids, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers for the pharmaceutical compositions of this invention. Suitable biodegradable microspheres are disclosed, for example, in U.S. Pat. Nos. 4,897,268 and 5,075,109.

Any of a variety of adjuvants may be employed in the vaccines of this invention to nonspecifically enhance the immune response. Suitable adjuvants are commercially available as, for example, Freund's Incomplete Adjuvant and Freund's Complete Adjuvant (Difco Laboratories) and Merck Adjuvant 65 (Merck and Company, Inc., Rahway, N.J.). Other suitable adjuvants include alum, biodegradable microspheres, monophosphoryl lipid A and quil A.

As noted above, polypeptides comprising an immunogenic portion of a CD4+ T cell stimulating antigen isolated using the methods of the present invention may also be employed in the diagnosis of disease, using techniques well known in the art. For example, polypeptides containing an immunogenic portion of a CD4+ T cell stimulating antigen isolated from an M. tuberculosis DNA expression library, as described in detail below, may be employed for the diagnosis of tuberculosis infection in a biological sample, using, for example, an ELISA technique. As used herein, a “biological sample” is any antibody-containing sample obtained from a patient. Preferably, the sample is whole blood, sputum, serum, plasma, saliva, cerebrospinal fluid or urine. More preferably, the sample is a blood, serum or plasma sample obtained from a patient or a blood supply.

The polypeptide(s) are used in an assay, to determine the presence or absence of antibodies to the polypeptide(s) in the sample, relative to a predetermined cut-off value. The presence of such antibodies indicates previous sensitization to the antigens which may be indicative of infection.

There are a variety of assay formats known to those of ordinary skill in the art for using one or more polypeptides to detect antibodies in a sample. See, e.g., Harlow and Lane, Antibodies: A Laboratory Manual, Cold Spring Harbor Laboratory, 1988, which is incorporated herein by reference. In a preferred embodiment, the assay involves the use of polypeptide immobilized on a solid support to bind to and remove the antibody from the sample. The bound antibody may then be detected using a detection reagent that contains a reporter group. Suitable detection reagents include antibodies that bind to the antibody/polypeptide complex and free polypeptide labeled with a reporter group (e.g., in a semi-competitive assay). Alternatively, a competitive assay may be utilized, in which an antibody that binds to the polypeptide is labeled with a reporter group and allowed to bind to the immobilized antigen after incubation of the antigen with the sample. The extent to which components of the sample inhibit the binding of the labeled antibody to the polypeptide is indicative of the reactivity of the sample with the immobilized polypeptide.

In certain embodiments, the assay is an enzyme linked immunosorbent assay (ELISA). This assay may be performed by first contacting a polypeptide that has been immobilized on a solid support, commonly the well of a microtiter plate, with the sample, such that antibodies to the polypeptide within the sample are allowed to bind to the immobilized polypeptide. Unbound sample is then removed from the immobilized polypeptide and a detection reagent capable of binding to the immobilized antibody-polypeptide complex is added. The amount of detection reagent that remains bound to the solid support is then determined using a method appropriate for the specific detection reagent. An appropriate detection reagent is any compound that binds to the immobilized antibody-polypeptide complex and that can be detected by any of a variety of means known to those in the art. Preferably, the detection reagent contains a binding agent (such as, for example, Protein A, Protein G, immunoglobulin, lectin or free antigen) conjugated to a reporter group. Preferred reporter groups include enzymes (such as horseradish peroxidase), substrates, cofactors, inhibitors, dyes, radionuclides, luminescent groups, fluorescent groups and biotin. The conjugation of binding agent to reporter group may be achieved using standard methods known to those of ordinary skill in the art. Common binding agents may also be purchased conjugated to a variety of reporter groups from many commercial sources (e.g., Zymed Laboratories, San Francisco, Calif., and Pierce, Rockford, Ill.).

The detection reagent is incubated with the immobilized antibody-polypeptide complex for an amount of time sufficient to detect the bound antibody. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of anti-polypeptide antibodies in the sample, the signal detected from the reporter group that remains bound to the solid support is generally compared to a signal that corresponds to a predetermined cut-off value.

The following Examples are offered by way of illustration and not by way of limitation.

### EXAMPLE 1

### Presentation of a Known CD4+ T Cell Antigen

The ability of the inventive method to identify CD4+ T cell-stimulating antigens was demonstrated as follows.

The known M. tuberculosis antigen Tb38-1, described in U. S. patent application Ser. No. 08/533,634, (SEQ ID NO:67) was inserted into a modified pBSK (pBluescript) vector (Stratagene, La Jolla, Calif.) having a six histide tag and an enterokinase site inserted in the polylinker, and transformed into E. coli. As a control, E. coli were transformed with empty vector. Protein expression was induced by the addition of isopropyl-β-D-thiogalactopyranoside (IPTG). The transformed E. coli were then incubated with either immature monocyte-derived dendritic cells or with adherent macrophages at 37° C. for between 60 to 90 minutes. Dendritic cells were prepared by culturing adherent PBMC for one week in GM-CSF and IL-4. The presence of peptide/MHC Class II complexes was determined by measuring proliferation and production of IFN-γ in a CD4+ T cell clone (referred to as 4E4) which was generated by limiting dilution analysis with a synthetic peptide from Tb38-1 using cells from a PPD-positive donor known to react with recombinant Tb38-1.

IFN-γ production was measured using an enzyme-linked immunosorbent assay (ELISA). ELISA plates were coated with a mouse monoclonal antibody directed to human IFN-γ (Chemicon, Temecula, Calif.) in PBS for four hours at room temperature. Wells were then blocked with PBS containing 5% (W/V) non-fat dried milk for 1 hour at room temperature. The plates were washed six times in PBS/0.2% TWEEN-20 and [samples] diluted 1:2 in culture medium, and the ELISA plates were incubated overnight at room temperature. The plates were again washed and a polyclonal rabbit anti-human IFN-γ serum diluted 1:4000 in PBS/10% normal goat serum was added to each well. The plates were then incubated for two hours at room temperature, washed and horseradish peroxidase-coupled anti-rabbit IgG (Jackson Laboratories, West Grove, Pa.) was added at a 1:4000 dilution in PBS/5% non-fat dried milk. After a further two hour incubation at room temperature, the plates were washed and substrate added. The reaction was stopped after 20 min with 1 N sulfuric acid. Optical density was determined at 450 nm using 570 nm as a reference wavelength.

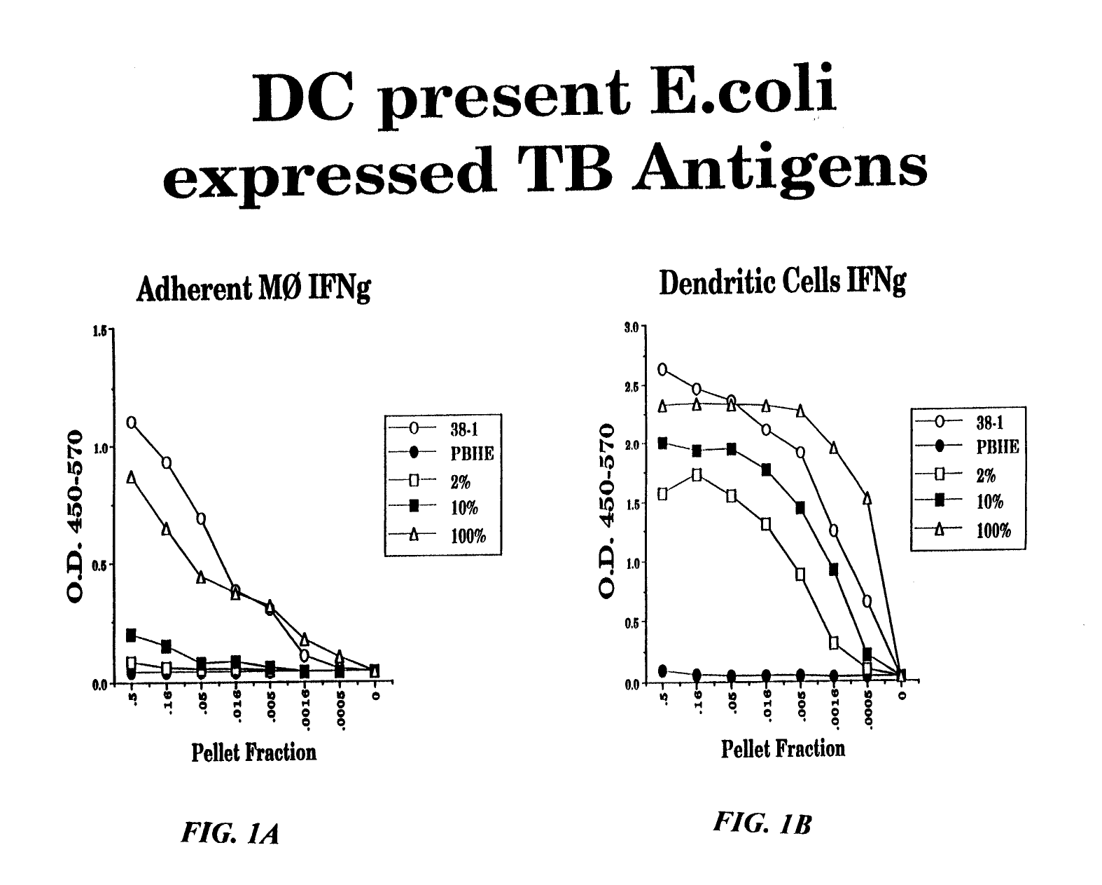

FIGS. 1A and B illustrate the presentation of E. coli-expressed Tb38-1 by macrophages and dendritic cells, respectively, to the TB38-1 reactive T cell clone 4E4, as measured by IFN-γ production. The induction of IFN-γ production by Tb38-1 transformed E. coli is compared with that for control E. coli (transformed with empty vector); Tb38-1 transformed E. coli diluted 1:10 with control E. coli (10%); Tb38-1 transformed E. coli diluted 1:50 with control E. coli (2%), and recombinant soluble Tb38-1 (1 μg/ml). Cultures were performed in duplicate in 96-well flat-bottom plates in a volume of 200 μl. Medium was RPMI/10% pooled human serum plus gentamicin.

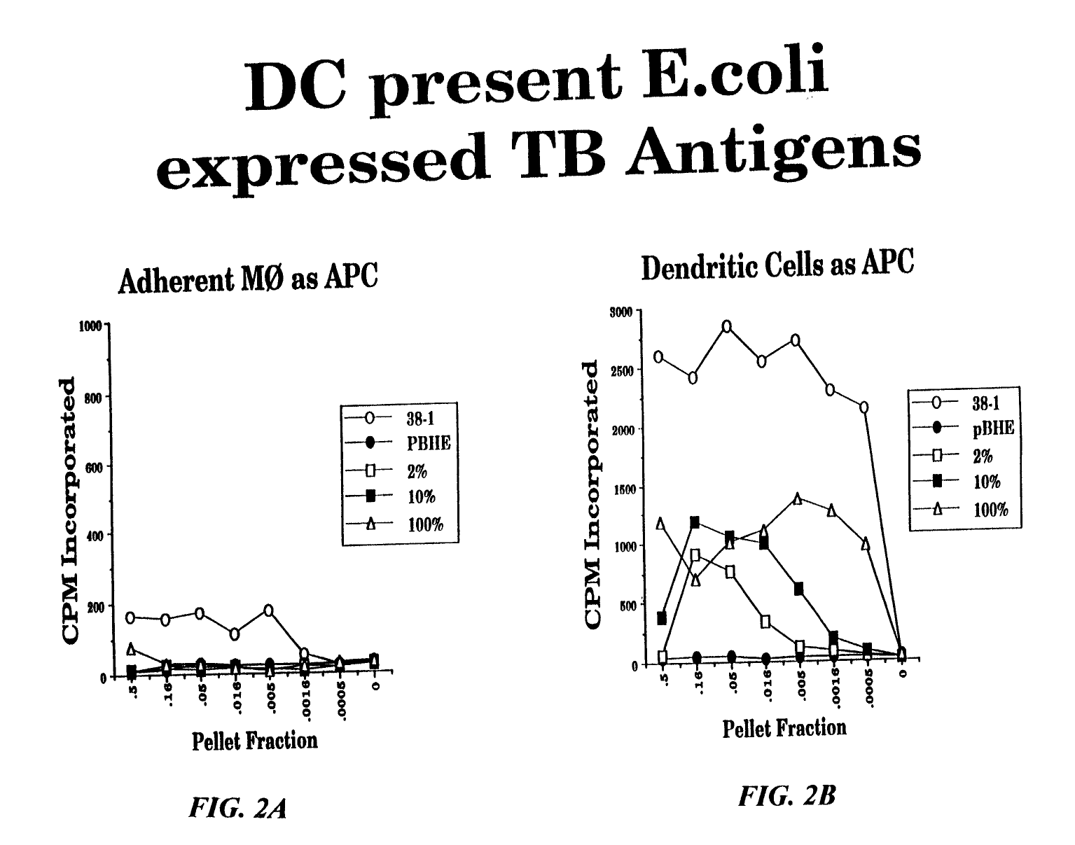

FIGS. 2A and B show data similar to FIGS. 1A and B, except that instead of production of interferon-γ, cell proliferation was measured. Specifically, FIGS. 2A and 2B illustrate the presentation of E. coli-expressed Tb38-1 by macrophages and dendritic cells, respectively, as measured by CD4+ T cell proliferation. The stimulation of cell proliferation by E. coli-expressed Tb38-1 is compared with that for: control E. coil (transformed with empty vector); Tb38-1 transformed E. coli diluted 1:10 with control E. coli (10%); and Tb38-1 transformed E. coli diluted 1:50 with control E. coli (2%). After three days of culture in 96-well round-bottom plates in a volume of 200 μl, the plates were pulsed with 1 μCi/well of tritiated thymidine for a further 18 hours, harvested and tritium uptake determined using a gas scintillation counter.

The ability of the inventive method to identify a CD4+ T cell-stimulating antigen when expressed by one of a multitude of E. coli colonies was demonstrated as follows. Two CD4+ T cell lines, referred to as DC-4 and DC-5, were generated against dendritic cells infected with M. tuberculosis. Specifically, dendritic cells were prepared from adherent PBMC from a single donor as described above and subsequently infected with tuberculosis. Lynphocytes from the same donor were cultured under limiting dilution conditions with the infected dendritic cells to generate the T cell lines DC-4 and DC-5. These cell lines were shown to react with crude soluble proteins from M. tuberculosis but not with Tb38-1. Limiting dilution conditions were employed to obtain a third CD4+ T cell line, referred to as DC-6, which was shown to react with both crude soluble proteins and Tb38-1.

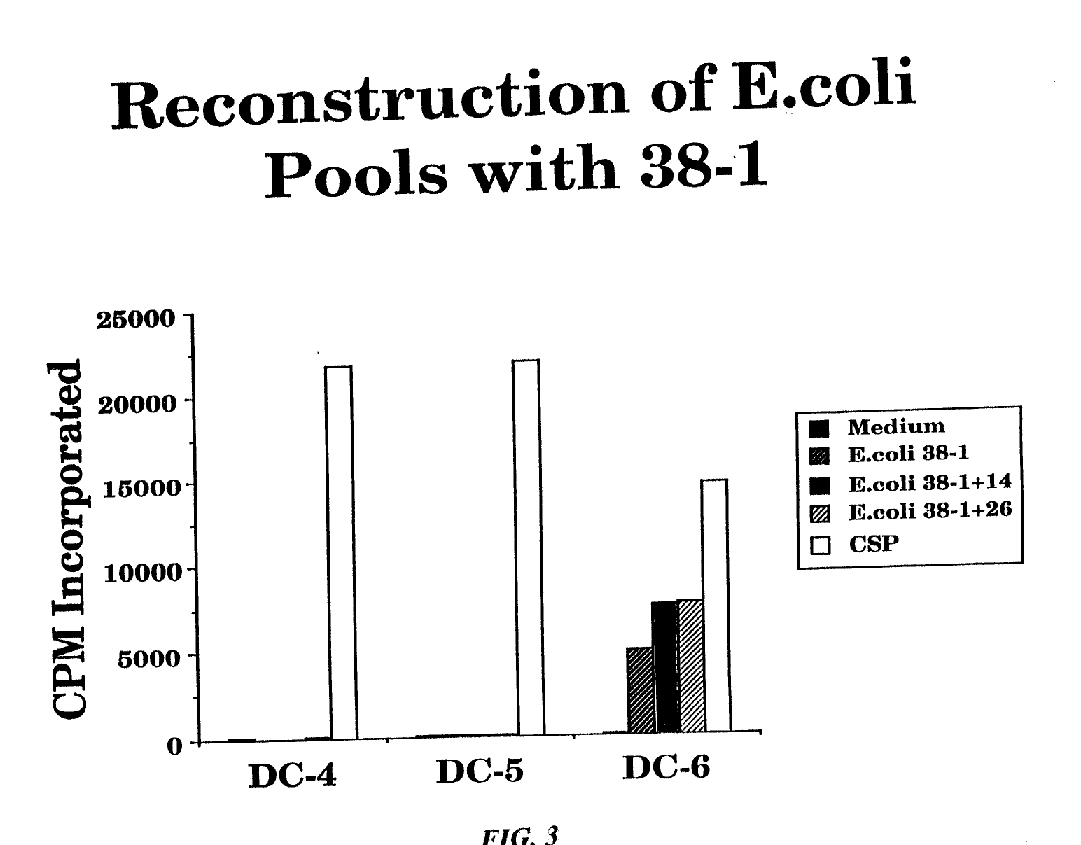

A single E. coli colony expressing the M. tuberculosis antigen Tb38-1 was mixed with either 0, 14 or 26 E. coli colonies expressing irrelevant antigens. These pools were grown overnight and then induced. The resulting pools were incubated with dendritic cells as described above and the presentation of peptide/MHC Class II complex was detected by measuring cell proliferation in the DC-4, DC-5 and DC-6 cell lines. The results are shown in FIG. 3, wherein CSP refers to culture supernatant M. tuberculosis proteins. Using the Tb38-1 specific T cell line, DC-6, the presence of E. coli-expressed Tb38-1 was detected in each of the E. coli pools. The T cell lines DC-4 and DC-5 did not react with E. coli-expressed Tb38-1.

Based upon the data shown in FIGS. 1-3, it was determined that a pool size of between about 30 and about 80, preferably about 50, is acceptable with moderate level expression of target antigen. However, use of a different expression vector may allow an increase in pool size.

### EXAMPLE 2

### Purification and Characterization of Novel CD4+ Antigens from M. Tuberculosis

Genomic DNA was isolated from the M. tuberculosis strains H37Rv and Erdman and used to construct expression libraries in the vector pBSK(−)using the Lambda ZAP expression system (Stratagene, La Jolla, Calif.). These libraries were transformed into E. coli, pools of induced E. coli cultures were incubated with dendritic cells, and the ability of the resulting incubated dendritic cells to stimulate cell proliferation and IFN-γ production in the CD4+ T cell line DC-6 was examined as described above. Positive pools were fractionated and re-tested until pure M. tuberculosis clones were obtained.

Twenty-three clones were isolated, of which nine were found to contain the previously identified M. tuberculosis antigens TbH-9 and Tb38-1, disclosed in U.S. patent application Ser. No. 08/533,634. The determined cDNA sequences for the remaining twelve clones (hereinafter referred to as Tb224, Tb636, Tb424, Tb436, Tb398, Tb508, Tb441, Tb475, Tb488, Tb465, Tb431 and Tb472) are provided in SEQ ID No: 1-12, respectively. The corresponding predicted amino acid sequences for Tb224, Tb636 and Tb431 are provided in SEQ ID NO: 13-15, respectively. These three antigens were found to show some homology to TbH-9, described above. Tb224 and Tb636 were found to be overlapping clones.

Tb424, Tb436, Tb398, Tb508, Tb441, Tb475, Tb488 and Tb465 were each found to contain two small open reading frames (referred to as ORF-1 and ORF-2) or truncated forms thereof, with minor variations in ORF-1 and ORF-2 being found for each clone. The predicted amino acid sequences of ORF-1 and ORF-2 for Tb424, Tb436, Tb398, Tb508, Tb441, Tb475, Tb488 and Tb465 are provided in SEQ ID NO: 16 and 17, 18 and 19, 20 and 21, 22 and 23, 24 and 25, 26 and 27, 28 and 29, and 30 and 31, respectively. In addition, clones Tb424 and Tb436 were found to contain a third apparent open reading frame, referred to as ORF-U. The predicted amino acid sequences of ORF-U for Tb424 and Tb436 are provided in SEQ ID NO: 32 and 33, respectively. Tb424 and Tb436 were found to be either overlapping clones or recently duplicated/transposed copies. Similarly Tb398, Tb508 and Tb465 were found to be either overlapping clones or recently duplicated/transposed copies, as were Tb475 and Tb488.

These sequences were compared with known sequences in the gene bank using the BLASTN system. No homologies to the antigens Tb224, Tb431 and Tb472 were found. Tb636 was found to be 100% identical to a cosmid previously identified in M. tuberculosis. Similarly, Tb508, Tb488, Tb398, Tb424, Tb436, Tb441, Tb465 and Tb475 were found to show homology to known M. tuberculosis cosmids. In addition, Tb488 was found to have 100% homology to M. tuberculosis topoisomerase I.

Seventeen overlapping peptides to the open reading frames ORF-1 (referred to as 1-1-1-17; SEQ ID NO: 34-50, respectively) and sixteen overlapping peptides to the open reading frame ORF-2 (referred to as 2-1-2-16, SEQ ID NO: 51-66) were synthesized using the procedure described below in Example 3.

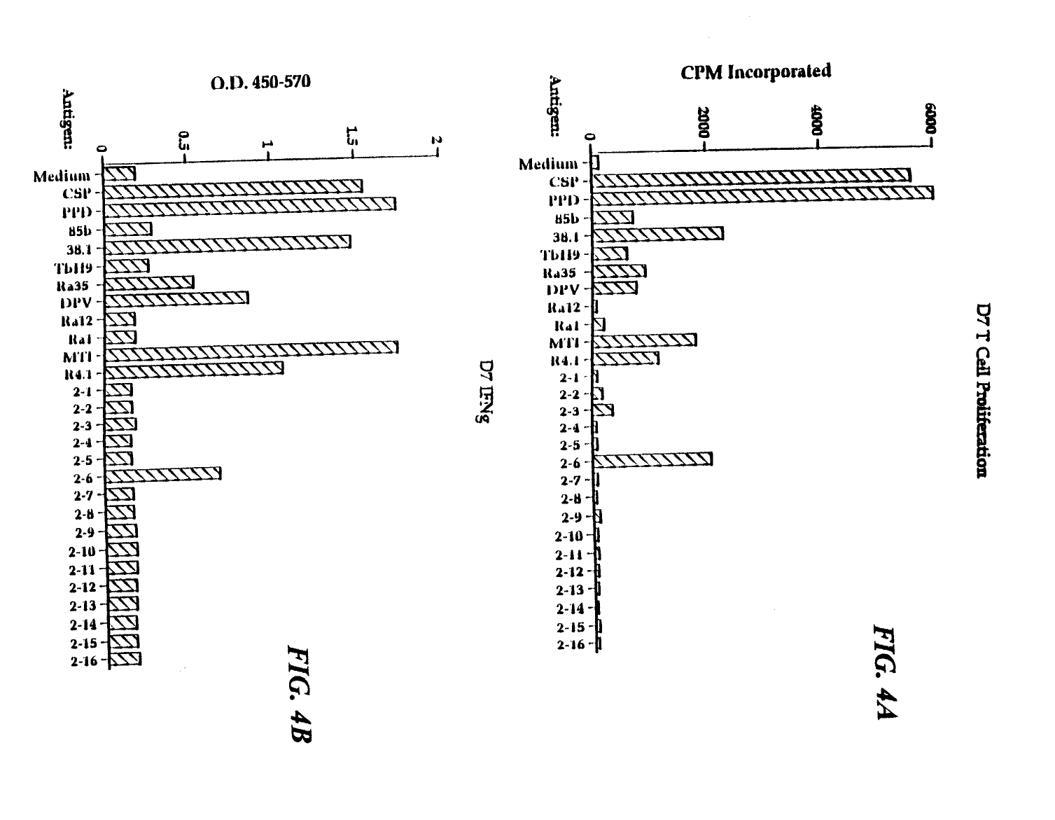

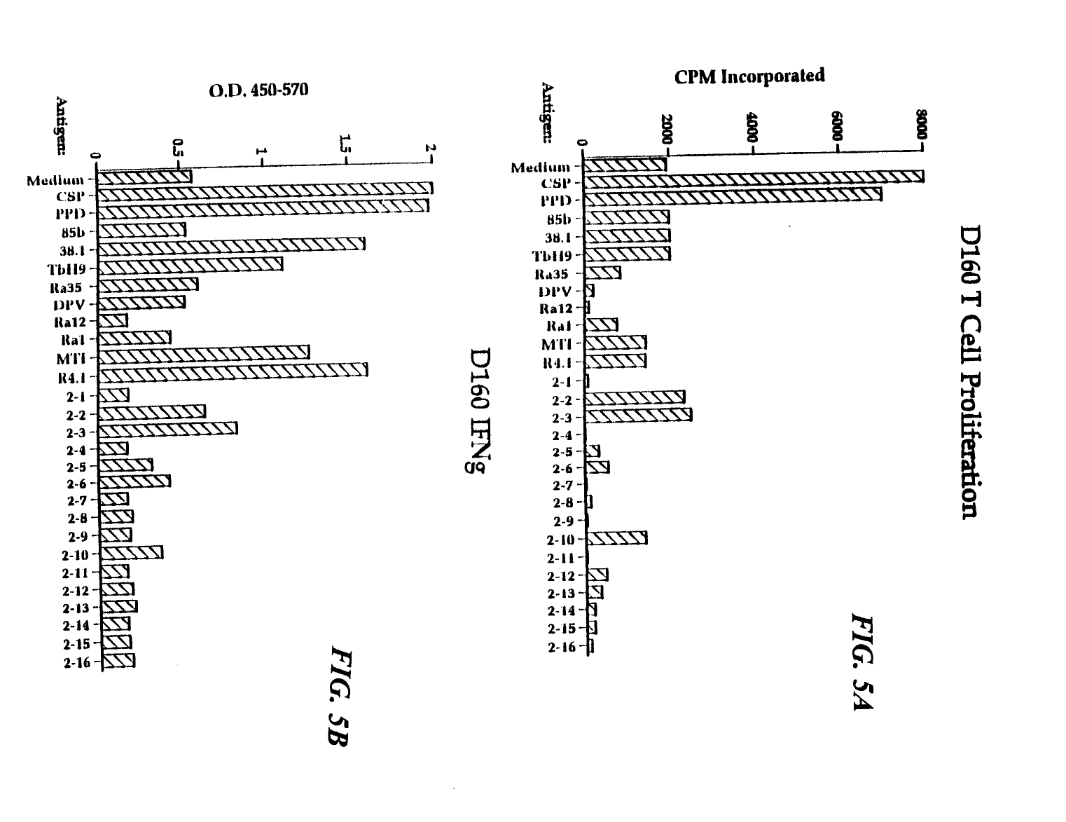

The ability of the synthetic peptides, and of recombinant ORF-1 and ORF-2, to induce T cell proliferation and IFN-γ production in PBMC from PPD-positive donors was assayed as described below in Example 4. FIGS. 4A-B illustrate stimulation of T cell proliferation and IFN-γ production, respectively, by recombinant ORF-2 (referred to as MTI) and the synthetic peptides 2-1-2-16 for a donor referred to as D7. FIGS. 5A-B illustrate stimulation of T cell proliferation and IFN-γ, respectively, for the donor D160 by recombinant ORF-2 and other known M. tuberculosis antigens. Recombinant ORF-2 stimulated T cell proliferation and IFN-γ production in PBMC from both donors. The amount of PBMC stimulation seen with the individual synthetic peptides varied with each donor, indicating that each donor recognizes different epitopes on ORF2.

Similarly, two overlapping peptides (SEQ ID NO: 68 and 69) to the open reading frame of Tb224 were synthesized and shown to induce T cell proliferation and IFN-γ production in PBMC from PPD-positive donors.

In subsequent studies, the above method was used to screen an M. tuberculosis genomic DNA library prepared as follows. Genomic DNA from M. tuberculosis Erdman strain was randomly sheared to an average size of 2 kb, and blunt ended with Klenow polymerase, followed by the addition of EcoRI adaptors. The insert was subsequently ligated into the Screen phage vector (Novagen, Madison, Wis.) and packaged in vitro using the PhageMaker extract (Novagen). The phage library (referred to as the Erd λScreen library) was amplified and a portion was converted into a plasmid expression library by an autosubcloning mechanism using the E. coli strain BM25.8 (Novagen). Plasmid DNA was purified from BM25.8 cultures containing the pSCREEN recombinants and used to transform competent cells of the expressing host strain BL21(DE3)pLysS. Transformed cells were aliquoted into 96 well microtiter plates with each well containing a pool size of approximately 50 colonies. Replica plates of the 96 well plasmid library format were induced with IPTG to allow recombinant protein expression. Following induction, the plates were centrifuged to pellet the E. coli which was used directly in T cell expression cloning of a T cell line prepared from a PPD-positive donor (donor 160) as described above. Pools containing E. coli expressing M. tuberculosis T cell antigens were subsequently broken down into individual colonies and reassayed in a similar fashion to identify positive hits.

Screening of the T cell line from donor 160 with one 96 well plate of the Erd λScreen library provided a total of nine positive hits. Previous experiments on the screening of the pBSK library described above with sera from donor 160 suggested that most or all of the positive clones would be TbH-9, Tb38-1 or MTI (disclosed in U.S. patent application Ser. No. 08/533,634) or variants thereof. However, Southern analysis revealed that only three wells hybridized with a mixed probe of TbH-9, Tb38-1 and MTI. Of the remaining six positive wells, two were found to be identical.

### EXAMPLE 3

### Synthesis of Synthetic Polypeptides

Polypeptides may be synthesized on a Millipore 9050 peptide synthesizer using FMOC chemistry with HPTU (O-Benzotriazole-N,N,N′,N′-tetramethyluronium hexafluorophosphate) activation. A Gly-Cys-Gly sequence may be attached to the amino terminus of the peptide to provide a method of conjugation or labeling of the peptide. Cleavage of the peptides from the solid support may be carried out using the following cleavage mixture: trifluoroacetic acid:ethanedithiol:thioanisole:water:phenol (40:1:2:2:3). After cleaving for 2 hours, the peptides may be precipitated in cold methyl-t-butyl-ether. The peptide pellets may then be dissolved in water containing 0.1% trifluoroacetic acid (TFA) and lyophilized prior to purification by C18 reverse phase HPLC. A gradient of 0-60% acetonitrile (containing 0.1% TFA) in water (containing 0.1% TFA) may be used to elute the peptides. Following lyophilization of the pure fractions, the peptides may be characterized using electrospray mass spectrometry and by amino acid analysis.

### EXAMPLE 4

### Induction of T Cell Proliferation abd Interferon-γ Production by M. Tuberculosis Antigens

The ability of recombinant M. tuberculosis antigens and synthetic peptides to induce T cell proliferation and interferon-γ production may be determined as follows.

Proteins may be induced by IPTG and purified by gel elution, as described in Skeiky et al. J. Exp. Med., 1995, 181:1527-1537. The purified polypeptides are then screened for the ability to induce T-cell proliferation in PBMC preparations. The PBMCs from donors known to be PPD skin test positive and whose T-cells are known to proliferate in response to PPD, are cultured in medium comprising RPMI 1640 supplemented with 10% pooled human serum and 50 μg/ml gentamicin. Purified polypeptides are added in triplicate at concentrations of 0.5 to 10 μg/mL. After five days of culture in 96-well round-bottom plates in a volume of 200 μl, 50 μl of medium is removed from each well for determination of IFN-γ levels, as described below. The plates are then pulsed with 1 μCi/well of tritiated thymidine for a further 18 hours, harvested and tritium uptake determined using a gas scintillation counter. Fractions that result in proliferation in all three replicates three fold greater than the proliferation observed in cells cultured in medium alone are considered positive.

IFN-γ is measured using an enzyme-linked immunosorbent assay (ELISA). ELISA plates are coated with a mouse monoclonal antibody directed to human IFN-γ (Chemicon, Temecula, Calif.) in PBS for four hours at room temperature. Wells are then blocked with PBS containing 5% (W/V) non-fat dried milk for 1 hour at room temperature. The plates are washed six times in PBS/0.2% TWEEN-20 and samples diluted 1:2 in culture medium in the ELISA plates are incubated overnight at room temperature. The plates are again washed and a polyclonal rabbit anti-human IFN-γ serum diluted 1:4000 in PBS/10% normal goat serum is added to each well. The plates are then incubated for two hours at room temperature, washed and horseradish peroxidase-coupled anti-rabbit IgG (Jackson Immunoresearch Laboratories, West Grove, Pa.) is added at a 1:4000 dilution in PBS/5% non-fat dried milk. After a further two hour incubation at room temperature, the plates are washed and TMB substrate added. The reaction is stopped after 20 min with 1 N sulfuric acid. Optical density is determined at 450 nm using 570 nm as a reference wavelength. Fractions that result in both replicates giving an OD two fold greater than the mean OD from cells cultured in medium alone, plus 3 standard deviations, are considered positive.

From the foregoing, it will be appreciated that, although specific embodiments of the invention have been described herein for the purpose of illustration, various modifications may be made without deviating from the spirit and scope of the invention.

1. A method for identifying DNA sequences that encode CD4+ T cell stimulating antigens, comprising:
a) transforming a host cell with a plasmid suspected of containing a DNA sequence that encodes a CD4+ T cell stimulating antigen and inducing antigen expression;
b) incubating the transformed host cell with at least one dendritic cell for a period of time sufficient to form an MHC II/peptide complex on the dendritic cell, wherein the peptide is derived from the expressed antigen;
c) incubating the dendritic cell with CD4+ T cells and determining the level of CD4+ T cell stimulation, thereby identifying a transformed host cell that expresses at least one CD4+ T cell stimulating antigen; and
d) isolating DNA from the transformed host cell.

2. The method of claim 1 wherein the level of CD4+ T cell stimulation is determined by measuring production of at least one cytokine.

3. The method of claim 2 wherein the cytokine is interferon-gamma.

4. The method of claim 1 wherein the level of CD4+ T cell stimulation is determined by measuring cell proliferation.

5. The method of claim 1 wherein the plasmid contains DNA isolated from an infectious disease agent.

6. The method of claim 5 wherein the infectious disease agent is M. tuberculosis.

7. The method of claim 1 wherein the plasmid contains DNA isolated from tumor tissue.

8. The method of claim 1 wherein the host cell is selected from the group consisting of E. coli, yeast and mammalian cells.

9. A method for identifying DNA sequences that encode CD4+ T cell stimulating antigens, comprising:
a) transforming a host cell with a plasmid suspected of containing a DNA sequence that encodes a CD4+ T cell stimulating antigen and inducing antigen expression;
b) incubating the transformed host cell with at least one dendritic cell for a period of time sufficient to form an MHC II/peptide complex on the dendritic cell, wherein the peptide is derived from the expressed antigen; and
c) incubating the dendritic cell with CD4+ T cells and determining the level of CD4+ T cell stimulation, thereby identifying a transformed host cell that expresses at least one CD4+ T cell stimulating antigen.

10. The method of claim 9 additionally comprising isolating DNA from the transformed host cell.

11. An isolated DNA sequence selected from the group consisting of:
a) DNA sequences isolated using the method of claim 1;
b) DNA sequences complementary to DNA sequences of a); and
c) DNA sequences that hybridize to a DNA sequence of a) or b) under moderately stringent conditions.

12. An expression vector comprising a DNA molecule according to claim 11.

13. A host cell transformed with an expression vector according to claim 12.

14. A polypeptide comprising an immunogenic portion of an antigen, or a variant of said antigen that differs only in conservative substitutions and/or modifications, the antigen comprising an amino acid sequence encoded by a DNA sequence according to claim 11.

15. A method for identifying DNA sequences that encode antigens comprising antibody epitopes, the method comprising:
a) transforming a host cell with a plasmid suspected of containing a DNA sequence that encodes a CD4+ T cell stimulating antigen and inducing antigen expression;
b) incubating the transformed host cell with at least one dendritic cell for a period of time sufficient to form an MHC II/peptide complex on the dendritic cell, wherein the peptide is derived from the expressed antigen;
c) incubating the dendritic cell with CD4+ T cells and determining the level of CD4+ T cell stimulation, thereby identifying a transformed host cell that expresses at least one CD4+ T cell stimulating antigen; and
d) isolating DNA from the transformed host cell.

16. The method of claim 51 wherein the level of CD4+ T cell stimulation is determined by measuring production of at least one cytokine.

17. The method of claim 16 wherein the cytokine is interferon-gamma.

18. The method of claim 15 wherein the level of CD4+ T cell stimulation is determined by measuring cell proliferation.

19. The method of claim 15 wherein the plasmid contains DNA isolated from an infectious disease agent.

20. The method of claim 19 wherein the infectious disease agent is M. tuberculosis.


SEQUENCE LISTING (69 sequences)

SEQ ID NO: 1 - DNA (genomic), 1886 bp, Mycobacterium tuberculosis; double-stranded, linear
cgctctggtgaccaccaacttcttcggtgtcaacaccatcccgatcgccctcaacgaggccgactacctgcgcatgtggatccaggccgccaccgtcatgagccactatcaagccgtcgcgcacgaaatctggtgtctccatgaatangccagttcgggaaagccgtgggccagtatcaccacgggtgcgccgggctcaccggcctcgaccactcgcagtcgcacgccgttggtatcaactaaccgtncngtangtgcgcccatcgtctcaccaaatcacaccgggcaccggcctgagaagggcttggggagcanccagaggcgattgtcgcgggtgctgccgcgcatcattgatcggccggccggaccantcgggcctcccttgacgtccggatcncacttcctgtgcagctggcatggctacagctcacagtgactgccccacgattgccggccaggtccagttcaaattccggtgaattcgcggacaaaagcagcaggtcaaccaaccgcagtcagtcgagggtcccaaacgtgagccaatcggtgaaatggcttgctgcagtgacaccggtcacaggcttagccgacagcaccggaatagctcaggcgggctatagagtcctatagaaacatttgctgatagaattaaccgctgtcttggcgtgatcttgatacggctcgccgtgcgaccggttggctcagtagctgaccaccatgtaacccatcctcggcaggtgtctactaaggcgagacaccgcattggtggggctgcatcgcaaatcggtccgagcatgtagcactgccgttatcccgggatagcaaaccacccggaaccagggctatcccagtcgctctccgacggaggccgtttcgctttccgttgcccgataactcccgagtggatatcggcgttatcanattcaggcttttcttcgcaaggtaccggtgttcgctatattcggatatctcggacggataattactaaaacttcagtggtttagataaggccgccgcaatacttcgccgatcttgccgagcgcaacggatttccatcgtcggttttcgtcgccttatcaaacatgatcggagataatgacagatcggcctagctaggtgtttagcggacgcgatttaggacaaccgagatttgctttgcctcgcaaccatgagagcgccccgcttcgacgccgaatcgggtgagtgatggtgggttagcacagccctgattgcgccaccggcgaggtgattgtgcccgccacgaggccgccgccggctagccccatgagcacgntatatagactctcctgcaacagatctcataccgatcgaaggcgaagcgcaggcatcgacgtcggagacactgccttgggatcgcgccgcctacacggcggttggcgcattgtcgcagcgcagttgcaggagggcaaatgtgcgcagacgatgtagtcgacaacaagtgnacatgccgtcttcacgaactcaaaactgacgatctgcttagcatgaaaaaaactgttgacatcggccaagcatgacagccagactgtaggcctacgcgtgcaatgcagaaccaaggntatgcatggaatcgacgaccgttgagataggcggcaggcatgagcagagcgttcatcatcgatccaacgatcagtgccattgacggcttgtacgaccttctggggattggaatacccaaccaagggggtatcctttactcctcactagagtacttcgaaaaagccctggaggagctggcagcagcgtttccgggtgatggctggttaggttcggccgcggacaaatacgccggcaaaaaccgcaaccacgtgaattttttccaggaactggcagacctcgatcgtcagctcatcagcctgatcca

SEQ ID NO: 2 - DNA (genomic), 2305 bp, Mycobacterium tuberculosis; double-stranded, linear
ggcacgcgctggccgcgcaatacaccgaaattgcaacggaactcgcaagcgtgctcgctgcggtgcaggcaagctcgtggcaggggcccagcgccgaccggttcgtcgtcgcccatcaaccgttccggtattggctaacccacgctgccacggtggccaccgcagcagccgccgcgcacnaaacggccgccgccgggtatacgtccgcattggggggcatgcctacgctagccgagttggcggccaaccatgccatgcacggcgctctggtgaccaccaacttcttcggtgtcaacaccatcccgatcgccctcaacgaggccgactacctgcgcatgtggatccaggccgccaccgtcatgagccactatcaagccgtcgcgcacgaaagcgtggcggcgacccccagcacgccgccggcgccgcagatagtgaccagtgcggccagctcggcggctagcagcagcttccccgacccgaccaaattgatcctgcagctactcaaggatttcctggagctgctgcgctatctggctgttgagctgctgccggggccgctcggcgacctcatcgcccaggtgttggactggttcatctcgttcgtgtccggtccagtcttcacgtttctcgcctacctggtgctggacccactgatctatttcggaccgttcgccccgctgacgagtccggtcctgttgcctgctgtggagttacgcaaccgcctcaaaaccgccaccggactgacgctgccacctaccgtgattttcgatcatcccactcccactgcggtcgccgagtatgtcgcccagcaaatgtctggcagccgcccaacggaatccggtgatccgacgtcgcaggttgtcgaacccgctcgtgccgaattcggcacgagtgctgttcatcaaatccccccgagacctgcggacacccggcgcgcttgccgacatcgagatgatgtcccgcgagatagcagaattgcccaacatcgtgatggtgcggggcttgacccgaccgaacggggaacctctgaaggagaccaaggtctcgtttcaggctggtgaagtgggcggcaagctcgacgaagcgaccaccctgctcgaagagcacggaggcgagctggaccagctgaccggcggtgcgcaccagttggccgacgccctcgcccaaatacgcaacgaaatcaatggggccgtggccagctcgagcgggatagtcaacaccctgcaggccatgatggacctgatgggcggtgacaagaccatccgacaactggaaaatgcgtcccaatatgtcgggcgcatgcgggctctgggggacaatctgagcgggaccgtcaccgatgccgaacaaatcgccacttgggccagccctatggtcaacgccctcaactccagcccggtgtgtaacagcgatcccgcctgtcggacgtcgcgcgcacagttggcggcgattgtccaggcgcaggacgacggcctgctcaggtccatcagagcgctagccgtcaccctgcaacagacgcaggaataccagacactcgcccggacggtgagcacactggacgggcaactgaagcaagtcgtcagcaccctcaaagcggtcgacggcctacccaccaaattggctcaaatgcagcaaggagccaacgctctcgccgacggcagcgcagcgctggcggcaggcgtgcaggaattggtcgatcaggtcaaaaagatgggctcagggctcaacgaggccgccgacttcctgttggggatcaagcgggatgcggacaagccgtcaatggcgggcttcaacattccaccgcagattttttcgagggacgagttcaagaagggcgcccagattttcctgtcggccgatggtcatgcggcgcggtacttcgtgcagagcgcgctgaatccggccaccaccgaggcgatggatcaggtcaacgatatcctccgtgttgcggattccgcgcgaccgaataccgaactcgaggatgccacgataggtctggcgggggttccgactgcgctgcgggatatccgcgactactacaacagcgatatgaaattcatcgtcattgcgacgatcgttatcgtattcttgattctcgtcattctgntgcgcgcacttgtggntccgatatatctgataggctcggtgctgatttcttacttgtcggccctaggcataggaactttcgttttccaattgatactgggccaggaaatgcattggagcctgccgggactgtccttcatattattggttgccatcggcgctgactacaacatgctgctcatttcacgcatccgcgacg

SEQ ID NO: 3 - DNA (genomic), 1742 bp, Mycobacterium tuberculosis; double-stranded, linear
ccgctctctttcaacgtcataagttcggtgggccagtcggccgcgcgtgcatatggcaccaataacgcgtgtcccatggatacccggaccgcacgacggtagagcggatcagcgcagccggtgccgaacactaccgcgtccacgctcagccctgccgcgttgcggaagatcgagcccaggttctcatggtcgttaacgccttccaacactgcgacggtgcgcgccccggcgaccacctgagcaacgctcggctccggcacccggcgcgcggctgccaacaccccacgattgagatggaagccgatcacccgtgccatgacatcagccgacgctcgatagtacggcgcgccgacaccggccagatcatccttgagctcggccagccggcggtcggtgccgaacagcgccagcggcgtgaaccgtgaggccagcatgcgctgcaccaccagcacaccctcggcgatcaccaacgccttgccggtcggcagatcgggacnacngtcgatgctgttcaggtcacggaaatcgtcgagccgtgggtcgtcgggatcgcagacgtcctgaacatcgaggccgtcggggtgctgggcacaacggccttcggtcacgggctttcgtcgaccagagccagcatcagatcggcggcgctgcgcaggatgtcacgctcgctgcggttcagcgtcgcgagccgctcagccagccactcttgcagagagccgttgctgggattaattgggagaggaagacagcatgtcgttcgtgaccacacagccggaagccctggcagctgcggcggcgaacctacagggtattggcacgacaatgaacgcccagaacgcggccgcggctgctccaaccaccggagtagtgcccgcagccgccgatgaagtatcagcgctgaccgcggctcagtttgctgcgcacgcgcagatgtaccaaacggtcagcgcccaggccgcggccattcacgaaatgttcgtgaacacgctggtggccagttctggctcatacgcggccaccgaggcggccaacgcagccgctgccggctgaacgggctcgcacgaacctgctgaaggagagggggaacatccggagttctcgggtcaggggttgcgccagcgcccagccgattcagntatcggcgtccataacagcagacgatctaggcattcagtactaaggagacaggcaacatggcctcacgttttatgacggatccgcatgcgatgcgggacatggcgggccgttttgaggtgcacgcccagacggtggaggacgaggctcgccggatgtgggcgtccgcgcaaaacatttccggtgcgggctggagtggcatggccgaggcgacctcgctagacaccatgacctagatgaatcaggcgtttcgcaacatcgtgaacatgctgcacggggtgcgtgacgggctggttcgcgacgccaacaantacgaacagcaagagcaggcctcccagcagatcctgagcagntagcgccgaaagccacagctgngtacgntttctcacattaggagaacaccaatatgacgattaattaccagttcggggacgtcgacgctcatggcgccatgatccgcgctcaggcggcgtcgcttgaggcggagcatcaggccatcgttcgtgatgtgttggccgcgggtgacttttggggcggcgccggttcggtggcttgccaggagttcattacccagttgggccgtaacttccaggtgatctacgagcagg

SEQ ID NO: 4 - DNA (genomic), 2836 bp, Mycobacterium tuberculosis; double-stranded, linear
gttgattccgttcgcggcgccgccgaagaccaccaactccgctggggtggtcgcacaggcggttgcgtcggtcagctggccgaatcccaatgattggtggctcngtgcggttgctgggctcgattacccccacggaaaggacgacgatcgttcgtttgctcggtcagtcgtacttggcgacgggcatggcgcggtttcttacctcgatcgcacagcagctgaccttcggcccagggggcacaacggctggctccggcggagcctggtacccaacgccacaattcgccggcctgggtgcaggcccggcggtgtcggcgagtttggcgcgggcggagccggtcgggaggttgtcggtgccgccaagttgggccgtcgcggctccggccttcgcggagaagcctgaggcgggcacgccgatgtccgtcatcggcgaagcgtccagctgcggtcagggaggcctgcttcgaggcataccgctggcgagagcggggcggcgtacaggcgccttcgctcaccgatacgggttccgccacagcgtgattacccggtctccgtcggcgggatagctttcgatccggtctgcgcggccgccggaaatgctgcagatagcgatcgaccgcgccggtcggtaaacgccgcacacggcactatcaatgcgcacggcgggcgttgatgccaaattgaccgtcccgacggggctttatctgcggcaagatttcatccccagcccggtcggtgggccgataaatacgctggtcagcgcgactcttccggctgaattcgatgctctgggcgcccgctcgacgccgagtatctcgagtgggccgcaaacccggtcaaacgctgttactgtggcgttaccacaggtgaatttgcggtgccaactggtgaacacttgcgaacgggtggcatcgaaatcaacttgttgcgttgcagtgatctactctcttgcagagagccgttgctgggattaattgggagaggaagacagcatgtcgttcgtgaccacacagccggaagccctggcagctgcggcggcgaacctacagggtattggcacgacaatgaacgcccagaacgcggccgcggctgctccaaccaccggagtagtgcccgcagccgccgatgaagtatcagcgctgaccgcggctcagtttgctgcgcacgcgcagatgtaccaaacggtcagcgcccaggccgcggccattcacgaaatgttcgtgaacacgctggtggccagttctggctcatacgcggccaccgaggcggccaacgcagccgctgccggctgaacgggctcgcacgaacctgctgaaggagagggggaacatccggagttctcgggtcaggggttgcgccagcgcccagccgattcagctatcggcgtccataacagcagacgatctaggcattcagtactaaggagacaggcaacatggcctcacgttttatgacggatccgcatgcgatgcgggacatggcgggccgttttgaggtgcacgcccagacggtggaggacgaggctcgccggatgtgggcgtccgcgcaaaacatttccggtgcgggctggagtggcatggccgaggcgacctcgctagacaccatgacctagatgaatcaggcgtttcgcaacatcgtgaacatgctgcacggggtgcgtgacgggctggttcgcgacgccaacaactacgaacagcaagagcaggcctcccagcagatcctgagcagctagcgccgaaagccacagctgcgtacgctttctcacattaggagaacaccaatatgacgattaattaccagttcggggacgtcgacgctcatggcgccatgatccgcgctcaggcggcgtcgcttgaggcggagcatcaggccatcgttcgtgatgtgttggccgcgggtgacttttggggcggcgccggttcggtggcttgccaggagttcattacccagttgggccgtaacttccaggtgatctacgagcaggccaacgcccacgggcagaaggtgcaggctgccggcaacaacatggcgcaaaccgacagcgccgtcggctccagctgggcctaaaactgaacttcagtcgcggcagcacaccaaccagccggtgtgctgctgtgtcctgcagttaactagcactcgaccgctgaggtagcgatggatcaacagagtacccgcaccgacatcaccgtcaacgtcgacggcttctggatgcttcaggcgctactggatatccgccacgttgcgcctgagttacgttgccggccgtacgtctccaccgattccaatgactggctaaacgagcacccggggatggcggtcatgcgcgagcagggcattgtcgtcaacgacgcggtcaacgaacaggtcgctgcccggatgaaggtgcttgccgcacctgatcttgaagtcgtcgccctgctgtcacgcggcaagttgctgtacggggtcatagacgacgagaaccagccgccgggttcgcgtgacatccctgacaatgagttccgggtggtgttggcccggcgaggccagcactgggtgtcggcggtacgggttggcaatgacatcaccgtcgatgacgtgacggtctcggatagcgcctcgatcgccgcactggtaatggacggtctggagtcgattcaccacgccgacccagccgcgatcaacgcggtcaacgtgccaatggaggagatctcgtgccgaattcggcacgaggcacgaggcggtgtcggtgacgacgggatcgatcacgatcatcgaccggccgggatccttggcgatctcgttgagcacgacccgggcccgcgggaagctctgcgacatccatgggttcttcccg

SEQ ID NO: 5 - DNA (genomic), 900 bp, Mycobacterium tuberculosis; double-stranded, linear
aacatgctgcacggggtgcgtgacgggctggttcgcgacgccaacaactacgagcagcaagagcaggcctcccagcagatcctcagcagctaacgtcagccgctgcagcacaatacttttacaagcgaaggagaacaggttcgatgaccatcaactatcagttcggtgatgtcgacgctcacggcgccatgatccgcgctcaggccgggttgctggaggccgaacatcaggccatcattcgtgatgtgttgaccgcgagtgacttttggggcggcgccggttcggcggcctgccaggggttcattacccaattgggccgtaacttccaggtgatctacgaacaggccaacgcccacgggcagaaggtgcaggctgccggcaacaacatggcgcaaaccgacagcgccgtcggctccagctgggcctgacaccaggccaaggccagggacgtggtgtacgagtgaaggttcctcgcgtgatccttcgggtggcagtctaggtggtcagtgctggggtgttggtggtttgctgcttggcgggttcttcggtgctggtcagtgctgctcgggctcgggtgaggacctcgaggcccaggtagcgccgtccttcgatccattcgtcgtgttgttcggcgaggacggctccgacgaggcggatgatcgaggcgcggtcggggaagatgcccacgacgtcggttcggcgtcgtacctctcggttgaggcgttcctgggggttgttggaccagatttggcgccagatcttcttggggaaggcggtgaacgccagcaggtcggtgcgggcggtgtcgangtgctcggccaccgcggggagtttgtcggtcagagcgtcgagtacccgatcatattgggcaacaactgattcggcgttgggctggtcgta

SEQ ID NO: 6 - DNA (genomic), 1905 bp, Mycobacterium tuberculosis; double-stranded, linear
gctcgccggatgtgggcgtccgcgcaaaacatttccggtgcgggctggagtggcatggccgaggcgacctcgctagacaccatggcccagatgaatcaggcgtttcgcaacatcgtgaacatgctgcacggggtgcgtgacgggctggttcgcgacgccaacaactacgagcagcaagagcaggcctcccagcagatcctcagcagctaacgtcagccgctgcagcacaatacttttacaagcgaaggagaacaggttcgatgaccatcaactatcagttcggtgatgtcgacgctcacggcgccatgatccgcgctcaggccgggttgctggaggccgagcatcaggccatcattcgtgatgtgttgaccgcgagtgacttttggggcggcgccggttcggcggcctgccaggggttcattacccagttgggccgtaacttccaggtgatctacgaacaagccaacacccacgggcagaaggtgcaagctgccggcaacaacatggcgcaaaccgacagcgccgtcngctccagctgggcctgacaccaggccaaggccagggacgtggtgtacnagtgaaggttcctcgcgtgatccttcgggtggcagtctaggtggtcagtgctggggtgttggtggtttgctgcttggcgggttcttcggtgctggtcagtgctgctcgggctcgggtgaggacctcgaggcccaggtagcgccgtccttcgatccattcgtcgtgttgttcggcgaggacngctccgacgangcggatgatcgaggcgcggtcggggaagatgcccacgacgtcggttcggcgtcgtacctctcggttgaagcgttcctgggggccaccgcttggcgccnangcactccacgccaattcgtcncacctaacagcggtggccaacgactatgactacgacaccgtttttgccagggccctcnaaaggatctgcgcgtcccggcgacacgctttttgcgataagtacctccggcaattctatgagtgtactgcggnccgcgaaaaccgcaagggagttgggtgtgacggttnttgcaaatgacgggcgaatccggcggccagctggcagaattcgcagatttcttgatcaacgtcccgtcacgcgacaccgggcgaatccaggaatctcacatcgtttttattcatgcgatctccgaacatgtcgaacacgcgcttttcgcgcctcgccaataggaaagccgatccttacgcggccattcgaaagatggtcgcggaacgtgcgggacaccaatggtgtctcttcctcgatagagacggggtcatcaatcgacaagtggtcggcgactacgtacggaactggcggcagtttgaatggttgcccggggcggcgcgggcgttgaagaagctacgggcatgggctccgtacatcgttgtcgtgacaaaccagcagggcgtgggtgccggattgatgagcgccgtcgacgtgatggtgatacatcggcacctccaaatgcagcttgcatccgatggcgtgctgatagatggatttcaggtttgcccgcaccaccgttcgcagcggtgtggctgccgtaagccgagaccgggtctggtcctcgactggctcggacgacaccccgacagtgagccattgctgagcatcgtggttggggacagcctcagcgatcttgacattggcacacaacgtcgccgctgctgccggtgcatgtgccagtgtccagatagggggcgccagttctggcggtgtcgctgacgcgtcatttgactcgctctgggagttcgctgtcgcagtcggacatgcgcggggggagcggggctaatggcgatcttgcgcgggcgagcgccgtngcggntcggactnngcggtggcgggacagacgtggaaccgtactcgagccagtt

SEQ ID NO: 7 - DNA (genomic), 2921 bp, Mycobacterium tuberculosis; double-stranded, linear
cgggatgccgtggtggttggtattgcccaaaccctggcgctggtccccggggtatccaggtccgggtcgaccatcagcgctggactgtttctcggactcgaccgtgaactggccgcccgattcggattcctgctggccattccagcggtgttcgcctccgggttgttctcgttgcccgacgcattccacccggtaaccgagggcatgagcgctactggcccgcagttgctggtggccaccctgatcgcgttcgtcctcggtctgaccgcggtggcctggctgctgcggtttctggtgcgacacaacatgtactggttcgtcggctaccgggtgctcgtcgggacgggcatgctcgtgctgctggctaccgggacggtagccgcgacatgaccgtcatcttgctacgccatgcccgttccacctcgaacaccgcgggcgtgctggccggccggtccggcgtcgacctcgacgagaaggggcgcgagcaggccaccgggttgatcgatcgaattggtgacctgccgatccgggcggtcgcgtcttctccaatgctgcggtgtcaacgcaccgtcgaaccgctggccgaggcgctgtgcctggagccgctcatcgatgaccggttctccgaagtcgactacggcgaatggactggcagaaaaatcggtgacctggtcgacgagccgttgtggcgggtagtccaggcccaccccagcgcggcggtgtttcccggcggtgagggtttggcgcaggtgcagacgtggttgtcctgacggatttccatgccggggaacaccaagaccggatcggcactggcggtcgccggcgaaaacccggccgccaatagggcgaccgtcgctgcgaatgcgcgtggtaccaggcggaccaccttgaactcccatccgtcggggccaagcgcatcgcccgccgccggttacggctaaggcgtaccaaaacccgacggtaatacttcggcaatgtcgggtcncgacgttaccgagacgtgaccagngaggcngcggcattggatttatcgatggtgcgcggttcccancccggcggtccgaanacgtagcccagccgatcccgcagacgtgttgccgaccgccagtcacgcacgatcgccacgtactcgcgggtctgcagcttccagatgttgaacgtgtcgacccgcttggtcaggccataatgcggtcggaatagctccggctgaaagctaccgaacaggcggtcccagatgatgaggatgccgccatagttcttgtccanatacaccgggtccattccgtggtggacccggtggtgcgacggggtattgaagacgaattcgaaccaccgcggcagcctgtcgatccgctcggtgtgcacccagaactggtagatcaagttcagcgaccaattgcagaacaccatccaagggggaagccccatcagtggcagcggaacccacatgagaatctcgccgctgttgttccantttctggcgcagcgcggtggcgaagttgaagtattcgctggagtgatgcgcctggtgggtagcccagatcagccgaactcggtgggcgatgcggtgataggagtagtacagcagatcgacaccaacgatcgcgatcacccaggtgtaccaccggtgggcggacagctgccagggggcaaggtaggcatagattgcggcataaccgagcagggcaagggacttccagccggcggtggtggctatcgaaaccagccccatcgagatgctggccaccgagtcgcgggtgaggtaagcgcccgaggcgggccgtggctgcccggtagcagcggtctcgatgctttccagcttgcgggccgccgtccattcgagaatcagcagcaatagaaaacatggaatggcgaacagtaccgggtcccgcatttcctcgggcagcgctgagaagaatccggcgacggcatggccgaggcgacctcgntagacaccatgacccagatgaatcaggcgtttcgcaacatcgtgaacatgctgcacggggtgcgtgacgggctggttcgcgacgccaacaantacgaacagcaagagcaggcctcccagcagatcctcagcagctgacccggcccgacgactcaggaggacacatgaccatcaactatcaattcggggacgtcgacgctcacggcgccatgatccgcgctcaggccgggtcgctggaggccgagcatcaggccatcatttctgatgtgttgaccgcgagtgacttttggggcggcgccggttcggcggcctgccaggggttcattacccagctgggccgtaacttccaggtgatntacgagcaggccaacgcccacgggcagaaggtgcaggctgccggcaacaacatggcacaaaccgacagcgccgtcggctccagctgggcataaagntggcttaaggcccgcgccgtcaattacaacgtggccgcacaccggttggtgtgtggccacgttgttatctgaacgactaactacttcgacctgctaaagtcggcgcgttgatccccggtcggatggtgctgaactgggaagatggcctcaatgcccttgttgcggaagggattgaggccatcgtgtttcgtactttaggcgatcagtgctggttgtgggagtcgctgctgcccgacgaggtgcgccgactgcccgaggaactggcccgggtggacgcattgttggacgatccggcgttcttcgccccgttcgtgccgttcttcgacccgcgcaggggccggccgtcgacgccgatggaggtctatctgcagttgatgtttgtgaagttccgctaccggctgggctatgagtcgctgtgccgggaggtggctgattcgatcacctgacggcggttttgccgcattgcgctggacgggtcggtgccgcatccgaccacattgatgaagctcaccacgcgttgc

SEQ ID NO: 8 - DNA (genomic), 1704 bp, Mycobacterium tuberculosis; double-stranded, linear
cgcgatcgtcgtcaacgangtcgaccgtcaccacggactgatcaacaagttcgcaggcgacgccgccctggccatcttcggagccccgaaccgcctcgaccgtcccgaagacgccgcgctggccgccgcccgggccatanccgancggctggccnacgagatgcccgaggtccaagccggcatcggggtggcggcaggccanatcgtcgccggcaatgtcggcgccaagcaaagattcnaatacacagtggtcggcaagccggtcaaccangcggcccgattgtgcgaactggccaaatcacaccccgcgcgattgggtctcgcccgctcggctcatggtcacccaattcaaggactactttggcctggcgcacgacctgccgaagtgggcgagtgaaggcgccaaagccgccggtgaggccgccaaggcgttgccggccgccgttccggccattccgagtgctggcctgagcggcgttgcgggcgccgtcggtcaggcggcgtcggtcgggggattgaaggttccggccgtttggaccgccacgaccccggcggcgagccccgcggtgctggcggcgtccaacggcctcggagccgcggccgccgctgaaggttcgacacacgcgtttggcgggatgccgctcatgggtancggtgccggacgtgcgtttaacaacttcgctgcccctcgatacggattcaagccgaccgtgatcgcccaaccgccggctggcggatgaccaactacgttcgttgatcgaggatcgaattcnacgattcaaagggaggaattcatatgacctcncgttttatgacggatccgcacgcnatncgggacatggcgggccgttttgaggtgcacgcccagacggtggaggacgaggctngcnggatgtgggcgtccgcgcaaaacatttccggtgcgggctggagtggcatggccgaggcgacctcgntagacaccatggcccagatgaatcaggcgtttcncaacatcgtgaacatgctgcacggggtgngtgacgggctggttcgcgacgccaacaactacgaacagcaagagcaggcctcccagcagatcctcagcagctgacccggcccgacgactcaggaggacacatgaccatcaactatcaattcggggacgtcgacgctcatggcgccatgatccgcgctntggccgggttgctggaggccgagcatcaggccatcatttctgatgtgttgaccgcgagtgacttttggggcggcgccggttcggcggcctgccaggggttcattacccagttgggccgtaacttccaggtgatttacgagcaggccaacgcccacgggcagaaggtgcaggctgccggcaacaacatggcacaaaccgacagcgccgtnggntccagctgggcctaacccgggtcntaagttgggtccgcgcagggcgggccgatcagcgtngactttggcgcccgatacacgggcatnttntngtcgggaacactgcgcccgcgtcagntgcccgcttccccttgttnggcgacgtgctcggtgatggctttgacgaccgcttcgccggcgcggccaatcaattggtcgcgcttgcctntagcccattcgtgcgacgcccgcggcgccgcgagttgtcccttgaaataaggaatcacagcacgggcgaacagctcataggagtgaaaggttgccgtggcggggccc

SEQ ID NO: 9 - DNA (genomic), 2286 bp, Mycobacterium tuberculosis; double-stranded, linear
ccgtcttggcgtctgggcgcattgtgatctgggccanttgcccctccacccagaccgcgcccagcttgtcgatccagcccgcgacccggattgccaccgcgcgaaccgggaacggattctccgctgaattctgggtcacttcgcagtcgcgcgggtgatcctgttggcgancagcgtctggaacgggcgtcnaacgcgtgccgtaagcccagcgtgtacgccgtcagcccgacgccgatgccgaatgccttgccgcccaagctgagccgcgcgggctccaccaagagcgtcacggtgagccagccaaccagatgcaaggcgacgatcaccgcgaagtgccgaattcggcacgagaggtgctggaaatccagcaatacgcccgcgagccgatctcgttggaccagaccatcggcgacganggcgacagncagcttggcgatttcatcgaaaacagcgaggcggtggtggncgtcgacgcggtgtccttcactttgctgcatgatcaactgcantcggtgctggacacgctctccgagcgtgaggcgggcgtggtgcggctacgcttcggccttaccgacggccagccgcgcacccttgacgagatcggccaggtctacggcgtgacccgggaacgcatccgccagatcgaatccaagactatgtcgaagttgcgccatccgagccgctcacaggtcctgcgcgactatcgtgccgaattcggcacgagccgttttgaggtgcacgcccagacggtggaggacgaggctcgccggatgtgggcgtccgcgcaaaacatttccggtgcgggctggagtggcatggccgangcgacctcgctagacaccatggcccagatgaatcaggcgtttcgcaacatcgtgaacatgctgcacggggtgcgtgacgggctggttcgcgacgccaacaactacgaacagcaagagcaggcctcccagcagatcctcagcagctgacccggcccgacgactcaggaggacacatgaccatcaactatcaattcggggacgtcgacgctcatggcgccatgatccgcgctctggccgggttgctggaggccgagcatcaggccatcatttctgatgtgttgaccgcgagtgacttttggggcggcgccggttcggcggcctgccaggggttcattacccagttgggccgtaacttccaggtgatctacgagcaggccaacgcccacgggcagaaggtgcaggctgccggcaacaacatggcacaaaccgacagcgccgtcggctccagctgggcctaacccgggtcctaagttgggtccgcgcagggcgggccgatcagcgtcgactttggcgcccgatacacgggcatgtngtngtcgggaacactgcgcccgcgtcagctgcccgcttccccttgttcggcgacgtgctcggtgatggctttgacgaccgcttcgccggcgcggccaatcaattggtcgcgcttgcctctagcctcgtgccgaattcggcacgagggtgctggtgccgcgctatcggcagcacgtgagctccacgacgaactcatcccagtgctgggttccgcggagttcggcatcggcgtgtcggccggaagggccatcgccggccacatcggcgctcaagcccgcttcgagtacaccgtcatcggcgacccggtcaacgaggccgcccggctcaccgaactggccaaagtcgaggatggccacgttctggcgtcggcgatcgcggtcagtggcgccctggacgccgaagcattgtgttgggatgttggcgaggtggttgagctccgcggacgtgctgcacccacccaactagccaggccaatgaatntggcngcacccgaagaggtttccagcgaagtacgcggctagtcgcgcttggctgcnttcttcgccggcaccttccgggcagctttcctggctggccgttttgccggaccccgggctcggcgatcggccaacagctcggcggcgcgctcgtcggttatggaagccacgtngtcgcccttacgcaggctggcattggtctcaccgtcggtgacgtacggcccgaatcggccgtccttgatgaccattggcttgccagacgccggatntgntcccagctcgcgcagcggcggagccgaagcgctttgccggccacgacntttcggctctgngtagatnttcagggcttcgtcgagcgngatggtgaatatatggtcttcggtgaccagtgatcgagaatcgttgccgcgctttagatacggtcngtagcgcccgttctgcgcggtgatntc

SEQ ID NO: 10 - DNA (genomic), 1136 bp, Mycobacterium tuberculosis; double-stranded, linear
gggcatcttccccgaccgcgcctcgatcatccgcctcgtcggagccgtcctcgccgaacaacacgacgaatggatcgaaggacggcgctacctgggcctcgaggtcctcacccgagcccgagcagcactgaccagcaccgaagaaccgccaagcagcaaaccaccaacaccccagcactgaccacctagactgccacccgaaggatcacgcgaggaaccttcactcgtacaccacgtccctggccttggcctggtgtcaggcccagctggagccgacggcgctgtcggtttgcgccatgttgttgccggcagcctgcaccttctgcccgtgggcgttggcctgctcgtagatcacctggaagttacggcccaactgggtaatgaacccctggcaggccgccgaaccggcgccgccccaaaagtcactcgcggtcaacacatcacgaatgatggcctgatgctcggcctccagcaacccggcctgagcgcggatcatggcgccgtgagcgtcgacatcaccgaactgatagttgatggtcatcgaacctgttctccttcgcttgtaaaagtattgtgctgcagcggctgacgttagctgctgaggatctgctgggaggcctgctcttgcctcgtgccgaattcggcacgagaggccgccttcgaagaaatcctttgagaattcgccaaggccgtcgacccagcatggggtcagctcgccagccgcgccggctggcaaccgttcccgctcgagaaagacctggaggaataccagtgacaaacgacctcccagacgtccgagagcgtgacggcggtccacgtcccgctcctcctgctggcgggccacgcttgtcagacgtgtgggtttacaacgggcgggcgtacgacctgagtgagtggatttccaagcatcccggcggcgccttnttcattgggcggaccaagaaccgcgacatcaccgcaatcgtcaagtcctaccatcgtgatccggcgattgtcgagcgaatcctgcagcggaggtacgcgttgggccgcgacgcaacccctagggacatccaccccaagcacaatgcaccggcatttctgttcaaagacgacttcaacagctggcgggacaccccgaagtatcgattngacga

SEQ ID NO: 11 - DNA (genomic), 967 bp, Mycobacterium tuberculosis; double-stranded, linear
tgagcgccaaccctaccgtcggttcgtcacacggaccgcatggcctgctccgcggactgccgctagggtcgcggatcactcggcgtagcggcgcctttgcccaccgatatgggttccgtcacagtgtggttgcccgcccgccatcggccggataacgccatgacctcagctcggcagaaatgacaatgctcccaaaggcgtgagcacccgaagacaactaagcaggagatcgcatgccgtttgtgactacccaaccagaagcactggcggcggcggccggcagtctgcagggaatcggctccgcattgaacgcccagaatgcggctgcggcgactcccacgacgggggtggtccggcggccgccgatgaantgtcggcgctgacggcggctcagttcgcggcacacgcccagatctatcaggccgtcagcgcccaggccgcggcgattcacgagatgttcgtcaacactctacagatgagctcagggtcgtatgctgctaccgaggccgccaacgcggccgcggccggntagaggagtcactgcgatggattttggggcgttgccgccggaggtcaattcggtgcggatgtatgccgttcctggctcggcaccaatggtcgctgcggcgtcggcctggaacgggttggccgcggagctgagttcggcggccaccggttatgagacggtgatcactcagctcagcagtgaggggtggctaggtccggcgtcagcggcgatggccgaggcagttgcgccgtatgtggcgtggatgagtgccgctgcggcgcaagccgagcaggcggccacacaggccagggccgccgcggccgcttttgaggcggcgtttgccgcgacggtgcctccgccgttgatcgcggccaaccgggcttcgttgatgcagctgatctcgacgaatgtctttggtcagaacacctcggcgatcgcggccgccgaagctcagtacgg

SEQ ID NO: 12 - DNA (genomic), 585 bp, Mycobacterium tuberculosis; double-stranded, linear
tggattccgatagcggtttcggcccctcgacgggcgaccacggcgcgcaggcctccgaacggggggccgggacgctgggattcgccgggaccgcaaccaaagaacgccgggtccgggcggtcgggctgaccgcactggccggtgatgagttcggcaacggcccccggatgccgatggtgccggggacctgggagcagggcagcaacgagcccgaggcgcccgacggatcggggagagggggaggcgacggcttaccgcacgacagcaagtaaccgaattccgaatcacgtggacccgtacgggtcgaaaggagagatgttatgagccttttggatgctcatatcccacagttggtggcctcccagtcggcgtttgccgccaaggcggggctgatgcggcacacgatcggtcaggccgagcaggcggcgatgtcggctcaggcgtttcaccagggggagtcgtcggcggcgtttcaggccgcccatgcccggtttgtggcggcggccgccaaagtcaacaccttgttggatgtcgcgcaggcgaatctgggtgaggccgccggtacctatgtggccgccgatgctg

SEQ ID NO: 13 - protein, 144 aa, Mycobacterium tuberculosis
ALVTTNFFGVNTIPIALNEADYLRMWIQAATVMSHYQAVAHEIWCLHEXASSGKPWASITTGAPGSPASTTRSRTPLVSTNRXVXAPIVSPNHTGHRPEKGLGSXQRRLSRVLPRIIDRPAGPXGPPLTSGSHFLCSWHGYSSQ

SEQ ID NO: 14 - protein, 352 aa, Mycobacterium tuberculosis
HALAAQYTEIATELASVLAAVQASSWQGPSADRFVVAHQPFRYWLTHAATVATAAAAAHXTAAAGYTSALGGMPTLAELAANHAMHGALVTTNFFGVNTIPIALNEADYLRMWIQAATVMSHYQAVAHESVAATPSTPPAPQIVTSAASSAASSSFPDPTKLILQLLKDFLELLRYLAVELLPGPLGDLIAQVLDWFISFVSGPVFTFLAYLVLDPLIYFGPFAPLTSPVLLPAVELRNRLKTATGLTLPPTVIFDHPTPTAVAEYVAQQMSGSRPTESGDPTSQVVEPARAEFGTSAVHQIPPRPADTRRACRHRDDVPRDSRIAQHRDGAGLDPTERGTSEGDQGLVSGW

SEQ ID NO: 15 - protein, 141 aa, Mycobacterium tuberculosis
MDFGALPPEVNSVRMYAVPGSAPMVAAASAWNGLAAELSSAATGYETVITQLSSEGWLGPASAAMAEAVAPYVAWMSAAAAQAEQAATQARAAAAAFEAAFAATVPPPLIAANRASLMQLISTNVFGQNTSAIAAAEAQYG

SEQ ID NO: 16 - protein, 58 aa, Mycobacterium tuberculosis
MASRFMTDPHAMRDMAGRFEVHAQTVEDEARRMWASAQNISGAGWSGMAEATSLDTMT

SEQ ID NO: 17 - protein, 67 aa, Mycobacterium tuberculosis
MTINYQFGDVDAHGAMIRAQAASLEAEHQAIVRDVLAAGDFWGGAGSVACQEFITQLGRNFQVIYEQ

SEQ ID NO: 18 - protein, 58 aa, Mycobacterium tuberculosis; identical to SEQ ID NO: 16

SEQ ID NO: 19 - protein, 94 aa, Mycobacterium tuberculosis
MTINYQFGDVDAHGAMIRAQAASLEAEHQAIVRDVLAAGDFWGGAGSVACQEFITQLGRNFQVIYEQANAHGQKVQAAGNNMAQTDSAVGSSWA

SEQ ID NO: 20 - protein, 30 aa, Mycobacterium tuberculosis
NMLHGVRDGLVRDANNYEQQEQASQQILSS

SEQ ID NO: 21 - protein, 94 aa, Mycobacterium tuberculosis
MTINYQFGDVDAHGAMIRAQAGLLEAEHQAIIRDVLTASDFWGGAGSAACQGFITQLGRNFQVIYEQANAHGQKVQAAGNNMAQTDSAVGSSWA

SEQ ID NO: 22 - protein, 69 aa, Mycobacterium tuberculosis
ARRMWASAQNISGAGWSGMAEATSLDTMAQMNQAFRNIVNMLHGVRDGLVRDANNYEQQEQASQQILSS

SEQ ID NO: 23 - protein, 94 aa, Mycobacterium tuberculosis
MTINYQFGDVDAHGAMIRAQAGLLEAEHQAIIRDVLTASDFWGGAGSAACQGFITQLGRNFQVIYEQANTHGQKVQAAGNNMAQTDSAVXSSWA

SEQ ID NO: 24 - protein, 52 aa, Mycobacterium tuberculosis
GMAEATSXDTMTQMNQAFRNIVNMLHGVRDGLVRDANXYEQQEQASQQILSS

SEQ ID NO: 25 - protein, 94 aa, Mycobacterium tuberculosis
MTINYQFGDVDAHGAMIRAQAGSLEAEHQAIISDVLTASDFWGGAGSAACQGFITQLGRNFQVXYEQANAHGQKVQAAGNNMAQTDSAVGSSWA

SEQ ID NO: 26 - protein, 98 aa, Mycobacterium tuberculosis
MTSRFMTDPHAMRDMAGRFEVHAQTVEDEARRMWASAQNISGAGWSGMAEATSLDTMAQMNQAFRNIVNMLHGVRDGLVRDANNYEQQEQASQQILSS

SEQ ID NO: 27 - protein, 94 aa, Mycobacterium tuberculosis
MTINYQFGDVDAHGAMIRAXAGLLEAEHQAIISDVLTASDFWGGAGSAACQGFITQLGRNFQVIYEQANAHGQKVQAAGNNMAQTDSAVGSSWA

SEQ ID NO: 28 - protein, 81 aa, Mycobacterium tuberculosis
RFEVHAQTVEDEARRMWASAQNISGAGWSGMAXATSLDTMAQMNQAFRNIVNMLHGVRDGLVRDANNYEQQEQASQQILSS

SEQ ID NO: 29 - protein, 94 aa, Mycobacterium tuberculosis
MTINYQFGDVDAHGAMIRALAGLLEAEHQAIISDVLTASDFWGGAGSAACQGFITQLGRNFQVIYEQANAHGQKVQAAGNNMAQTDSAVGSSWA

SEQ ID NO: 30 - protein, 11 aa, Mycobacterium tuberculosis
QEQASQQILSS

SEQ ID NO: 31 - protein, 94 aa, Mycobacterium tuberculosis; identical to SEQ ID NO: 21

SEQ ID NO: 32 - protein, 99 aa, Mycobacterium tuberculosis
MSFVTTQPEALAAAAANLQGIGTTMNAQNAAAAAPTTGVVPAAADEVSALTAAQFAAHAQMYQTVSAQAAAIHEMFVNTLVASSGSYAATEAANAAAAG

SEQ ID NO: 33 - protein, 99 aa, Mycobacterium tuberculosis; identical to SEQ ID NO: 32

SEQ ID NO: 34 - protein, 15 aa, Mycobacterium tuberculosis
DPHAMRDMAGRFEVH

SEQ ID NO: 35 - protein, 15 aa, Mycobacterium tuberculosis
RDMAGRFEVHAQTVE

SEQ ID NO: 36 - protein, 15 aa, Mycobacterium tuberculosis
RFEVHAQTVEDEARR

SEQ ID NO: 37 - protein, 15 aa, Mycobacterium tuberculosis
AQTVEDEARRMWASA

SEQ ID NO: 38 - protein, 15 aa, Mycobacterium tuberculosis
DEARRMWASAQNISG

SEQ ID NO: 39 - protein, 15 aa, Mycobacterium tuberculosis
MWASAQNISGAGWSG

SEQ ID NO: 40 - protein, 15 aa, Mycobacterium tuberculosis
QNISGAGWSGMAEAT

SEQ ID NO: 41 - protein, 16 aa, Mycobacterium tuberculosis
AGWSGMAEATSLDTMT

SEQ ID NO: 42 - protein, 15 aa, Mycobacterium tuberculosis
MAEATSLDTMAQMNQ

SEQ ID NO: 43 - protein, 15 aa, Mycobacterium tuberculosis
SLDTMAQMNQAFRNI

SEQ ID NO: 44 - protein, 15 aa, Mycobacterium tuberculosis
AQMNQAFRNIVNMLH

SEQ ID NO: 45 - protein, 15 aa, Mycobacterium tuberculosis
AFRNIVNMLHGVRDG

SEQ ID NO: 46 - protein, 15 aa, Mycobacterium tuberculosis
VNMLHGVRDGLVRDA

SEQ ID NO: 47 - protein, 15 aa, Mycobacterium tuberculosis
GVRDGLVRDANNYEQ

SEQ ID NO: 48 - protein, 15 aa, Mycobacterium tuberculosis
LVRDANNYEQQEQAS

SEQ ID NO: 49 - protein, 16 aa, Mycobacterium tuberculosis
NNYEQQEQASQQILSS

SEQ ID NO: 50 - protein, 17 aa, Mycobacterium tuberculosis
MASRFMTDPHAMRDMAG

SEQ ID NO: 51 - protein, 15 aa, Mycobacterium tuberculosis
MTINYQFGDVDAHGA

SEQ ID NO: 52 - protein, 15 aa, Mycobacterium tuberculosis
QFGDVDAHGAMIRAQ

SEQ ID NO: 53 - protein, 15 aa, Mycobacterium tuberculosis
DAHGAMIRAQAASLE

SEQ ID NO: 54 - protein, 15 aa, Mycobacterium tuberculosis
MIRAQAASLEAEHQA

SEQ ID NO: 55 - protein, 15 aa, Mycobacterium tuberculosis
AASLEAEHQAIVRDV

SEQ ID NO: 56 - protein, 15 aa, Mycobacterium tuberculosis
AEHQAIVRDVLAAGD

SEQ ID NO: 57 - protein, 15 aa, Mycobacterium tuberculosis
IVRDVLAAGDFWGGA

SEQ ID NO: 58 - protein, 16 aa, Mycobacterium tuberculosis
LAAGDFWGGAGSVACQ

SEQ ID NO: 59 - protein, 15 aa, Mycobacterium tuberculosis
FWGGAGSVACQEFIT

SEQ ID NO: 60 - protein, 15 aa, Mycobacterium tuberculosis
GSVACQEFITQLGRN

SEQ ID NO: 61 - protein, 18 aa, Mycobacterium tuberculosis
QEFITQLGRNFQVIYEQA

SEQ ID NO: 62 - protein, 15 aa, Mycobacterium tuberculosis
RNFQVIYEQANAHGQ

SEQ ID NO: 63 - protein, 15 aa, Mycobacterium tuberculosis
IYEQANAHGQKVQAA

SEQ ID NO: 64 - protein, 15 aa, Mycobacterium tuberculosis
NAHGQKVQAAGNNMA

SEQ ID NO: 65 - protein, 15 aa, Mycobacterium tuberculosis
KVQAAGNNMAQTDSA

SEQ ID NO: 66 - protein, 16 aa, Mycobacterium tuberculosis
GNNMAQTDSAVGSSWA

SEQ ID NO: 67 - DNA (genomic), 1617 bp, Mycobacterium tuberculosis; double-stranded, linear
catcggagggagtgatcaccatgctgtggcacgcaatgccaccggagntaaataccgcacggctgatggccggcgcgggtccggctccaatgcttgcggcggccgcgggatggcagacgctttcggcggctctggacgctcaggccgtcgagttgaccgcgcgcctgaactctctgggagaagcctggactggaggtggcagcgacaaggcgcttgcggctgcaacgccgatggtggtctggctacaaaccgcgtcaacacaggccaagacccgtgcgatgcaggcgacggcgcaagccgcggcatacacccaggccatggccacgacgccgtcgctgccggagatcgccgccaaccacatcacccaggccgtccttacggccaccaacttcttcggtatcaacacgatcccgatcgcgttgaccgagatggattatttcatccgtatgtggaaccaggcagccctggcaatggaggtctaccaggccgagaccgcggttaacacgcttttcgagaagctcgagccgatggcgtcgatccttgatcccggcgcgagccagagcacgacgaacccgatcttcggaatgccctcccctggcagctcaacaccggttggccagttgccgccggcggctacccagaccctcggccaactgggtgagatgagcggcccgatgcagcagctgacccagccgctgcagcaggtgacgtcgttgttcagccaggtgggcggcaccggcggcggcaacccagccgacgaggaagccgcgcagatgggcctgctcggcaccagtccgctgtcgaaccatccgctggctggtggatcaggccccagcgcgggcgcgggcctgctgcgcgcggagtcgctacctggcgcaggtgggtcgttgacccgcacgccgctgatgtctcagctgatcgaaaagccggttgccccctcggtgatgccggcggctgctgccggatcgtcggcgacgggtggcgccgctccggtgggtgcgggagcgatgggccagggtgcgcaatccggcggctccaccaggccgggtctggtcgcgccggcaccgctcgcgcaggagcgtgaagaagacgacgaggacgactgggacgaagaggacgactggtgagctcccgtaatgacaacagacttcccggccacccgggccggaagacttgccaacattttggcgaggaaggtaaagagagaaagtagtccagcatggcagagatgaagaccgatgccgctaccctcgcgcaggaggcaggtaatttcgagcggatctccggcgacctgaaaacccagatcgaccaggtggagtcgacggcaggttcgttgcagggccagtggcgcggcgcggcggggacggccgcccaggccgcggtggtgcgcttccaagaagcagccaataagcagaagcaggaactcgacgagatctcgacgaatattcgtcaggccggcgtccaatactcgagggccgacgaggagcagcagcaggcgctgtcctcgcaaatgggcttctgacccgctaatacgaaaagaaacggagcaaaaacatgacagagcagcagtggaatttcgcgggtatcgaggccgcggcaagcgcaatccagggaaat

SEQ ID NO: 68 - protein, 25 aa, Mycobacterium tuberculosis
VTTNFFGVNTIPIALNEADYLRMWI

SEQ ID NO: 69 - protein, 25 aa, Mycobacterium tuberculosis
NEADYLRMWIQAATVMSHYQAVAHE